(19) United States
(12) Patent Application Publication
(10) Pub. No.: US 2012/0058935 A1
(43) Pub. Date: Mar. 8, 2012

(54) ANTIMICROBIAL COMPOSITIONS AND METHODS OF USE THEREFORE

(75) Inventors: Robert Moir, Medfield, MA (US); Rudolph E. Tanzi, Hull, MA (US)

(73) Assignee: The General Hospital Corporation, Boston, MA (US)

(21) Appl. No.: 13/256,171

(22) PCT Filed: Mar. 12, 2010
(86) PCT No.: PCT/US10/27186

§ 371 Date: Nov. 18, 2011

Related U.S. Application Data
(60) Provisional application No. 61/159,671, filed on Mar. 12, 2009.

(51) Int. Cl. A61K 38/17; A61P 31/04; A01P 1/00; A61P 31/12; A01P 3/00; A01N 37/18; A61P 31/10

(52) U.S. Cl. 514/2.6; 514/2.3; 514/2.4; 514/3.3; 514/3.7; 514/3.4; 514/2.8; 514/2.7

(57) ABSTRACT

The invention features antimicrobial compositions comprising β-amyloid peptides, oligomers, and analogs thereof, and methods of using them for the prevention or treatment of an infection.

### CROSS-REFERENCE TO RELATED APPLICATION

This application claims the benefit of the following U.S. Provisional Application No. 61/159,671, filed Mar. 12, 2009, the entire contents of which are incorporated herein by reference.

### BACKGROUND OF THE INVENTION

The amyloid β-protein (Aβ) is believed to be the key mediator of Alzheimer's disease (AD) pathology. Aβ is most often characterized as an incidental catabolic byproduct that lacks a normal physiological role. However, Aβ has been shown to be a specific ligand for a number of different receptors and other molecules, transported by complex trafficking pathways, modulated in response to a variety of environmental stressors, and able to induce pro-inflammatory activities.

### SUMMARY OF THE INVENTION

As described below, the present invention features antimicrobial compositions and methods of using them for the prevention or treatment of an infection.

In one aspect, the invention provides a method for treating or preventing a microbial infection in a subject in need of treatment or prevention involving contacting the subject with an effective amount of one or more of a β-amyloid peptide, oligomer, derivative or analog thereof, thereby preventing or inhibiting a microbial infection in the subject.

In another aspect, the invention provides a method for treating or preventing a skin infection in a subject involving administering to the subject an effective amount of one or more of a β-amyloid peptide, oligomer, derivative or analog thereof, in an amount sufficient to treat the subject.

In yet another aspect, the invention provides a method for preventing or inhibiting microbial growth on a surface, involving contacting the surface with an effective amount of one or more of a β-amyloid peptide, oligomer, derivative or analog thereof, thereby preventing or inhibiting microbial growth on the surface.

In still another aspect, the invention provides a method of sanitizing or disinfecting a body part involving contacting the body part with an effective amount of a β-amyloid peptide, oligomer, derivative or analog thereof, thereby sanitizing or disinfecting the body part.

In one aspect, the invention provides an antimicrobial composition containing an effective amount of a β-amyloid peptide, oligomer, derivative or analog thereof, thereof in a carrier or diluent.

In another aspect, the invention provides a pharmaceutical pack containing a β-amyloid peptide, oligomer, derivative or analog thereof, in an individual dosage amount.

In various embodiments of any of the aspects delineated herein, the effective amount comprises 1% to 90% of a β-amyloid peptide, oligomer, derivative or analog thereof, or combinations thereof. In various embodiments of any of the aspects delineated herein, the β-amyloid peptide, oligomer, derivative or analog thereof, is formulated as a water soluble or water insoluble solid, liquid, emulsion, slurry, or powder. In various embodiments, the composition is topically administered.

In various embodiments of any of the aspects delineated herein, the β-amyloid peptide, oligomer, derivative or analog thereof, is formulated as a skin cleanser. In various embodiments of any of the aspects delineated herein, the skin is cleansed in preparation for surgery.

In various embodiments of any of the aspects delineated herein, the microbe is a bacteria, virus, or fungus. In various embodiments of any of the aspects delineated herein, microbe is one or more of Candida albicans, Escherichia coli, Staphylococcus epidermidis, Streptococcus pneumoniae, Staphylococcus aureus, Listeria monocytogenes, Enterococcus faecalis, Streptococcus agalactiae Pseudomonas aeruginosa, Streptococcus pyogenes, Streptococcus mitis, and Streptococcus salivariu.

In various embodiments of any of the aspects delineated herein, the method cleans, sanitizes, or disinfects the surface. In various embodiments, the surface is a wall, floor, ceiling, counter, machine, or other surface or equipment present in an industrial, commercial, or clinical setting. In various embodiments, the clinical setting is a hospital, medical office, clinic, health center, or laboratory. In other embodiments, the surface contacted is present in a dwelling, school, office building, plane, train, automobine, restaurant, cafeteria, or daycare center.

The invention provides antimicrobial compositions and methods of using them for the prevention or treatment of an infection. Compositions and articles defined by the invention were isolated or otherwise manufactured in connection with the examples provided below. Other features and advantages of the invention will be apparent from the detailed description, and from the claims.

### DEFINITIONS

By “β-amyloid peptide” is meant a 4.2-kD polypeptide having a 37-43-amino acid sequence present in amyloid plaques and soluble aggregates, which accumulate in the brains of patients with Alzheimer's disease. Exemplary Aβ peptides include peptides having the following amino acid sequences:

DAEFRHDSGYEVHHQKLVFFAEDVGSNKGAIIGLMVGGVVIAT
(Aβ43);
DAEFRHDSGYEVHHQKLVFFAEDVGSNKGAIIGLMVGGVVIA (Aβ42);
DAEFRHDSGYEVHHQKLVFFAEDVGSNKGAIIGLMVGGVV (Aβ40);
DAEFRHDSGYEVHHQKLVFFAEDVGSNKGAIIGLMVG (Aβ37).

By “anti-microbial composition” is meant any composition that prevents, inhibits, slows, or reduces the growth, proliferation, or survival of a microbe (e.g., bacteria, fungus (e.g., mold), protozoa, virus).

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “body part” is meant any tissue or organ of a mammalian or avian organism. Exemplary body parts include, but are not limited to, skin, hair, fur, epidermis, feathers, wool, hide, the oral cavity, including but not limited to, the lips, tongue, and teeth.

By “dwelling” is meant any human residence.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include bacterial invasion or colonization of a host cell.

By “effective amount” is meant an amount of a compound that prevents, inhibits, slows, stabilizes, or reduces the growth of a microbe. An effective amount of the compound described above may range from about 0.1 ppm to about 1000 ppm. Effective amounts may vary depending on the microbe to be inhibited, the application method, as well as the possibility of co-usage with other agents.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “industrial antimicrobial” is meant an antimicrobial composition used to clean, sanitize, or disinfect a surface in any industrial setting. In various embodiments, industrial antimicrobials are used on walls, floors, ceilings, counters, and machinery present in factories, food processing facilities, and hospitals.

By “inhibiting microbial growth” is meant preventing, slowing, or otherwise reducing the proliferation of a microbe. Such inhibition is by at least about 5%, 10%, 25%, 50%, 75%, or 100%.

By “home antimicrobial” is meant an antimicrobial composition used to clean, sanitize, or disinfect a surface with a home. In various embodiments, home antimicrobials are used in a kitchens, bathrooms, and other rooms within a dwelling to prevent or inhibit microbial growth on walls, floors, carpets, ceilings, counters, appliances, food preparation or storage vessels, or other implements used in food preparation.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “microbe” is meant a bacterium, fungus, protozoa, virus, or other microscopic organism. Microbes include airborne pathogens.

By “pathogen” is meant any bacteria, viruses, fungi, or protozoans capable of interfering with the normal function of a cell.

Exemplary bacterial pathogens include, but are not limited to, Aerobacter, Aeromonas, Acinetobacter, Agrobacterium, Bacillus, Bacteroides, Bartonella, Bordtella, Brucella, Burkholderia, Calymmatobacterium, Campylobacter, Citrobacter, Clostridium, Cornyebacterium, Enterobacter, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia.

By “protective immune response” is meant an immune response sufficient to ameliorate a pathogen infection in a mammal.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a graph showing that growth of E. faecalis is inhibited by Aβ42. E. faecalis were cultured alone (circle) with 25 μg/ml of Aβ42 (triangle) or LL-37 (diamond). Bacterial growth with time was monitored by inoculation of agar with diluted incubants and counting CFU. Representative data from six experiments is shown as mean signal of four replicates±s.e.m. At the time points indicated in the graph, incubants were monitored for Aβ42 and LL-37 by Western blot with mAb 6E10 or anti-LL-37. The figure shows representative signal for Aβ42 (odd lanes) or LL-37 (even lanes) incubants from six replicate experiments.

FIGS. 2A and 2B are images showing that E. faecalis pre-incubated with Aβ42 are mAb 6E10 immunoreactive. Bacteria were incubated (1 hr at 37° C.) with (FIG. 2A) or without (FIG. 2B) Aβ42 (25 μg/ml). Following repeated washes, the bacteria were fixed onto glass slides and immunostained with the HRP conjugated anti-Aβ antibody (mAb 6E10-HRP).

FIGS. 3A and 3B are graphs showing that AD brain homogenates have increased antimicrobial activity against C. albicans. AD and non-AD brain samples were tested for Aβ-mediated inhibition of C. albicans. Samples of temporal lobe (Temp. L.) and cerebellum (Cereb.) from AD (n=32) and age-matched control subjects (n=13) were homogenized in culture broth. In FIG. 3A, homogenates were inoculated with log-phase C. albicans and microbial growth determined by alamar blue viability assay. Data is shown as percentage of signal for C. albicans alone (average of four replicates)±s.e.m. In FIG. 3B, homogenates were assayed for Aβ40 and Aβ42 by commercially available ELISA. The graph in FIG. 3B shows Aβ signal (sum of Aβ40 and Aβ42) against C. albicans growth for temporal lobe homogenates from combined AD and non-demented cohorts (n=42). Probability analysis used unpaired two-tailed t-tests (p). Correlation was determined by calculating the Pearson r correlation coefficient (r).

FIGS. 4A and 4B depict results showing that immunodepletion of Aβ from AD brain homogenates attenuates C. albicans inhibition. Homogenates of temporal lobe (Temp. L.) and cerebellum (Cereb.) were prepared from AD (n=32) or non-demented (n=13) subjects. AD (AD) or non-demented (non-AD) homogenates were pooled and then incubated with Magno-beads pre-loaded with rabbit IgG (IgG) or a polyconal rabbit anti-Aβ antibody (α-Aβ). Following bead removal samples were analyzed for Aβ signal by Western blot and assayed for C. albicans growth by alamar blue viability assay. FIG. 4A shows C. albicans growth in treated homogenates as a percentage of signal in culture broth alone. Immunodepletion of AD temporal lobe homogenates with α-Aβ restored microbial growth to levels equivalent to non-demented control samples. Graph shows average of five replicates±s.e.m. Statistical probability analysis (p) of data used unpaired two-tailed t-test. In FIG. 4B, untreated and immunodepleted homogenates (1:16 dilution) were Western blotted and probed with the Aβ-specific mAb 4G8 antibody. Analysis confirmed Aβ signal was reduced in temporal lobe homogenate incubated with anti-amyloid β-peptide antibody (Lane 1) compared to sample incubate alone (Lane 2) or with rabbit IgG (Lane 3). Aβ in dilutions of cerebellum homogenate is below the level of detection for our experimental conditions (Lanes 4-6). Blots included synthetic Aβ42 (Aβ42) standard (Lane 7).

FIG. 5 is a graph showing that Aβ-mediated inhibition of C. albicans in AD brain homogenates is dose dependant. AD temporal lobe (Temp. L.) or cerebellum (Cereb.) were homogenized in phosphate buffer. Temporal lobe (n=30.) or cerebellum (n=32) homogenates were pooled and 1:16, 1:32, and 1:64 serial dilutions prepared in culture broth. Homogenate dilutions were incubated with mouse IgG (IgG) or anti-Aβ mAb 6E10 (6E10) antibody immobilized on MagnaBind beads. Following pelleting of the beads incubants were inoculated with mid-logarithmic phase C. albicans in 96-well plates. Microbial growth was determined by alamar blue cell viability assay. Graphs shows percentage signal of C. albicans alone (average of five replicates)±s.e.m. Consistent with Aβ-mediated antimicrobial activity, C. albicans growth is highest for samples with low Aβ levels and increases with homogenate dilution.

### DETAILED DESCRIPTION OF THE INVENTION

The invention features antimicrobial compositions and related prophylactic and therapeutic methods.

The invention is based, at least in part, on the discovery that amyloid β-protein (Aβ) is a hitherto unrecognized antimicrobial peptide (AMPs) that normally functions in the innate immune system. This finding stands in stark contrast to current models of Aβ-mediated pathology and has important implications for ongoing and future Alzheimer disease treatment strategies. As reported in more detail below, in vitro assays were used to compare the antimicrobial activities of Aβ and LL-37, an archetypical human AMP. Aβ was found to have antimicrobial activity against eight common and clinically relevant microorganisms with a potency equivalent to, and in some cases greater than, LL-37. Furthermore, AD whole brain homogenates have significantly higher antimicrobial activity than aged matched non-AD samples and AMP action correlates with tissue Aβ levels. Consistent with Aβ-mediated activity, the increased antimicrobial action was ablated by immunodepletion of AD brain homogenates with anti-Aβ antibodies.

### Amyloid β-Peptide

The past 25 years has witnessed the accrual of a large body of data concerning the physiochemistry and biological activities of the amyloid β-peptide (Aβ), the main component of β-amyloid deposits in the brains of Alzheimer's disease (AD) patients. Aβ, which is generated in the brain and peripheral tissues, is widely believed to be an incidental catabolic byproduct of the amyloid β protein precursor (APP) with no normal physiological function. Aβ has been shown to be a ligand for a number of different receptors and other molecules [2,3,4], transported by complex trafficking pathways between tissues and across the blood brain barrier [1,5], modulated in response to a variety of environmental stressors, and able to induce pro-inflammatory activities [6,7]. Nevertheless, the normal physiological role of Aβ remains unknown. Many of the physiochemical and biological properties previously reported for Aβ are similar to those of a group of biomolecules collectively known as “antimicrobial peptides” (AMPs) which function in the innate immune system. AMPs (also called “host defense peptides”) are potent, broad-spectrum antibiotics that target Gram-negative and Gram-positive bacteria, mycobacteria, enveloped viruses, fungi, protozoans and in some cases, transformed or cancerous host cells. AMPs are also potent immunomodulators that mediate cytokine release and adaptive immune responses (see review by Zaiou, 2007 [8]).

The three main families of mammalian AMPs are the defensins, the histatins, and the cathelicidins. Only one member of the cathelicidin family has been identified in humans, the LL-37 peptide [9]. The pleiotropic LL-37 peptide is a widely expressed archetypal AMP [10]. The rodent LL-37 homologue (CRAMP) has been shown to play a central role in combating bacterial infections in a range of tissues, including the CNS [11]. Patients that express low levels of LL-37 are at increased risk for serious infections [12]. Conversely, high levels of LL-37 are associated with the pathology of several presumably non-infectious diseases [13], including plaques in atherosclerosis [14]. As reported in more detail below, LL-37 exhibits striking similarities to Aβ, including a propensity to form cytotoxic soluble oligomers [15,16,17,18] and insoluble fibrils that demonstrate congophilia and birefringence [19], two classical histochemical properties of tinctorial amyloid. While the microbiocidal activity of LL-37 has been well characterized [20], the activity of Aβ against microbial organisms has not been tested.

As reported herein, Aβ is active against at least eight common and clinically relevant microorganisms. The in vitro antimicrobial activity of Aβ matched, and in some cases, exceeded, that of LL-37, an archetypical human AMP. Furthermore, anti-Aβ immunoreactive material in AD whole brain homogenates is active against Candida albicans, the pathogen identified herein as most sensitive to synthetic A13. Most strikingly, temporal lobe samples from AD brain contained significantly higher antimicrobial activity than material from the same brain area of aged-matched, non-AD subjects. Consistent with an Aβ-mediated action, cerebellum samples with low β-amyloid loads from the same set of affected and unaffected subjects were not significantly different with regards to antimicrobial activity. These findings show Aβ possesses antimicrobial activity and may function in vivo as an AMP and, thus, play a role as an effector molecule of innate immunity.

Accordingly, the invention features antimicrobial compositions containing β amyloid peptides and methods employing such compositions for the treatment and prevention of pathogen infections.

### Amyloid Beta Peptide and Analogs

Also included in the invention are beta amyloid peptides, oligomers, and fragments thereof that are modified in ways that enhance or do not inhibit their antimicrobial activity. In one embodiment, the invention provides methods for optimizing an β amyloid peptide amino acid sequence or nucleic acid sequence by producing an alteration. Such changes may include certain mutations, deletions, insertions, or post-translational modifications. The invention further includes analogs of any naturally-occurring polypeptide of the invention. Analogs can differ from the naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 10, 13, 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., B or γ amino acids.

In addition to full-length polypeptides, the invention also includes fragments of any one of the polypeptides of the invention. As used herein, the term “a fragment” means at least 5, 10, 13, or 15. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein β amyloid peptide analogs having a chemical structure designed to mimic β amyloid peptides functional activity can be administered according to methods of the invention. β amyloid peptide analogs may exceed the antimicrobial activity of native beta amyloid peptides. Methods of analog design are well known in the art, and synthesis of analogs can be carried out according to such methods by modifying the chemical structures such that the resultant analogs exhibit the antimicrobial activity of a native beta amyloid peptide. These chemical modifications include, but are not limited to, substituting alternative R groups and varying the degree of saturation at specific carbon atoms of the native β amyloid peptide molecule. Preferably, the β amyloid peptide analogs are relatively resistant to in vivo degradation, resulting in a more prolonged therapeutic effect upon administration. Assays for measuring functional activity include, but are not limited to, those described below and in the Examples.

### Anti-Microbial Activity

The invention provides compositions comprising β-amyloid peptides, oligomers, and analogs thereof for the treatment or prevention of infections with any of a number of pathogens, including but not limited to bacteria, viruses, protists, and fungi. In one embodiment, the invention provides for the treatment or prevention of bacterial infections, including infections with gram negative and gram positive bacteria, which serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Burkholderia sps, Borellia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia ssp, Yersinia pestis and Actinomyces israelli.

In other embodiments, the invention provides for the treatment or prevention of a protist infection. Such organisms include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

In still other embodiments, the invention provides for the treatment or prevention of an infection with a pathogenic fungus, including, without limitation, Alternaria, Aspergillus, Basidiobolus, Bipolaris, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, and Candida lusitaniae, Coccidioides, Cladophialophora, Cryptococcus, Cunninghamella, Curvularia, Exophiala, Fonsecaea, Histoplasma, Madurella, Malassezia, Plastomyces, Rhodotorula, Scedosporium, Scopulariopsis, Sporobolomyces, Tinea, and Trichosporon.

Fungi, including, but not limited to Candida, cause invasive diseases in hosts with altered immunity, such as patients with HIV infection, organ or bone marrow transplants, or neutropenia following cancer immunotherapy. There are approximately 200 species of the genus Candida, but nine cause the great majority of human infections. They are C. albicans, C. krusei, C. glabrata (formerly called Torulopsis glabrata), C. parapsilosis, C. tropicalis, C. pseudotropicalis, C. guilliermondii, C. dubliniensis, and C. lusitaniae. They cause infections of the mucous membranes, for example, thrush, esophagitis, and vagititis; skin, for example, intertrigo, balanitis, and generalized candidiasis; blood stream infections, for example, candidemia; and deep organ infections, for example, hepatosplenic candidiasis, urinary tract candidiasis, arthritis, endocarditis, and endophthamitis.

In still other embodiments, the methods of the invention can be used to treat or prevent a viral infection. Accordingly, the invention provides a composition comprising a β-amyloid peptides, oligomers, and analogs thereof that inhibits the growth of a viral pathogen. Exemplary viral pathogens include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

The present invention further provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to an infectious disease or disorder or symptom thereof. The method includes the step of administering to the mammal a therapeutic amount of a compound herein (e.g., β-amyloid and analogs thereof) sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae (e.g., β-amyloid, oligomers thereof, and analogs thereof) herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which an infectious pathogen or other microbe may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a pathogen infection, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Skin Infections

Compositions of the invention are particularly useful as antimicrobials to prevent, slow, or otherwise inhibit the growth of a pathogen effecting the skin. Preferably, compositions of the invention are used topically. For example, compositions of the invention can be formulated for the treatment or prevention of pathogen infections of the skin, including the treatment of bacterial infections. The most common bacterial skin bacteria are Staphylococcus aureus and group A b-hemolytic streptococci. Bacterial infections of the skin include infections by e.g., Mycobacterium tuberculosis, Micrococcus luteus, Propionibacterium acnes, Staphylococcus aureus, Streptococcus pyogenes, and Staphylococcus epidermidis. Bacterial infections can result in a variety of skin conditions, including impetigo, which is associated with S. aureus or S. pyogenes, erysipelas, which is associated with Streptococcus or Staphylococcus, and cellulitis, which is associated with S. aureus or S. pyogenes infection. Folliculitis, furuncles, and carbuncles are infections associated with S. aureus. Compounds of the invention have natural antibiotic and/or bacteriostatic activities, which are useful for the treatment or prevention of skin conditions associated with bacteria or a bacterial infection.

β-amyloid peptides, oligomers, and analogs thereof are typically present in a diluent or carrier at levels ranging from about 0.1% to about 95%. The methods herein contemplate administration of an effective amount of compound or compound composition comprising β-amyloid peptides, oligomers, and analogs thereof to achieve the desired or stated antibiotic or bacteriostatic effect. Preferably, the amount of active ingredient is combined with carrier materials to form a composition (e.g., cream, lotion, powder, topical spray) suitable for application to the skin. For some applications, anti-inflammatory, antibiotic or bacteriostatic compositions of the invention are formulated for topical application.

A typical anti-inflammatory, antibiotic or bacteriostatic formulation will contain from about 1% to about 95% β-amyloid peptides, oligomers, and analogs thereof, where the bottom of the range is any integer between 5 and 94 and the top of the range is any integer between 6 and 95, where the β-amyloid peptides, oligomers, and analogs thereof are provided in a carrier that is suitable for topical application. Where antibiotic or bacteriostatic compositions are desired, the compositions of the invention are preferably formulated with a carrier suitable for topical administration. The ratio of β-amyloid peptides, oligomers, and analogs thereof to carrier ranges between about 1:2 and 1:100. Preferred ratios include 1:2, 1:3, 1:4, 1:5, 1:6, 1:7, 1:8, 1:9, 1:10, 1:20, 1:30, 1:50, 1:75, and 1:100. Alternatively, compositions of the invention include at least about 1%, 10%, 20%, 30%, 50%, 60%, 75%, 80%, 90%, or 95% β-amyloid peptides, oligomers, and analogs thereof in a diluent or carrier.

In preferred embodiments, the preparation includes between 1 and 95% (e.g., 1, 2, 3, 4, 5, 6, 7, 8, 9, 10, 25, 75, 80, 90, or 95%) β-amyloid peptides, oligomers, and analogs thereof in a carrier or diluent. Alternatively, such preparations contain from about 20% to about 80% β-amyloid peptides, oligomers, and analogs thereof. Compositions containing β-amyloid peptides, oligomers, and analogs thereof are manufactured by ordinary methods. β-amyloid peptides, oligomers, and analogs thereof suitable for addition to products can be formulated as ordinary tablets, capsules, solids, liquids, emulsions, slurries, fine granules or powders, which are suitable for administration to products during their preparation, following preparation but prior to storage, or at any time prior to their sale to a vendor or consumer. Lower or higher amounts than those recited above may be required. The compositions delineated herein include the compounds of the formulae delineated herein, as well as additional anti-inflammatory, antibiotic or bacteriostatic activity agents if present, in amounts effective for inhibiting an inflammatory process, bacterial growth or reducing bacterial proliferation. Compositions of the invention are used to prevent, reduce, inhibit, slow or stabilize the growth, proliferation, or survival of a bacterium (e.g., . . . , Micrococcus luteus, E. faecalis, Propionibacterium acnes, Staphylococcus aureus, Streptococcus pyogenes, and Staphylococcus epidermidis).

Lower or higher doses than those recited herein may be required to effectively inhibit an epidermal bacterium (e.g., . . . , Micrococcus luteus, E. faecalis, Propionibacterium acnes, Staphylococcus aureus, Streptococcus pyogenes, and Staphylococcus epidermidis). Specific dosage and treatment regimens are determined empirically as described herein. Compositions of the invention are also useful for preventing the establishment of a skin condition, such as a skin bacterial infection and for maintaining or enhancing the health and/or appearance of skin.

### Dermatologic Formulations

Compositions of the invention containing β-amyloid peptides, oligomers, and analogs thereof are useful not only for promoting the health and condition of skin, but also for enhancing the appearance of skin. Accordingly, the invention provides for cosmetic and dermatological preparations of various forms. Preferably, the present invention provides personal care compositions that include an effective amount of a combination of compounds of the invention, with the balance of the composition comprising one or more compounds from the group of carriers, excipients, liposomes, active ingredients, biological or botanical products, humectants, emollients, surfactants, thickening agents, silicone components, organic sunscreens, preservatives, neutralizing agents, perfumes or pigments. Such compositions improve or enhance skin health, condition, or appearance, and are provided, for example, as a cream, lotion, liquid, solution, an anhydrous preparation, an oil-free preparation, an emulsion or microemulsion, a gel, a solid stick, a wipe, a patch, an ointment, or as an aerosol. It may also be advantageous to administer compositions of the invention in encapsulated form, for example in collagen matrices or in other conventional encapsulation materials, for example as cellulose encapsulations, in gelatine, wax matrices, or liposomally encapsulated.

In one approach, compositions of the invention are provided in a personal care composition, such as a moisturizing body wash, body wash, antimicrobial cleanser, skin protective cream, body lotion, facial cream, moisturizing cream, facial cleansing emulsion, surfactant-based facial cleanser, facial exfoliating gel, anti-acne treatment, facial toner, exfoliating cream, facial mask, after-shave balm or radioprotective. Skin care compositions include topically applied over-the-counter compositions, anti-fungal treatments, anti-acne treatments, skin protectants, and antiperspirants.

The invention also provides methods for making personal care compositions comprising combining an effective amount of a β-amyloid peptides, oligomers, and analogs thereof composition of the invention with a physiologically acceptable carrier or excipient to provide a personal care composition. The type of carrier utilized in the present invention depends on the type of product form desired for the personal care composition. In some embodiments, the carrier is a solid, while in other embodiments, it is semi-solid or liquid. Suitable carriers include liquids, as well as semi-solids (e.g., creams, lotions, gels, sticks, ointments, pastes, sprays and mousses). In particular, carriers that are lotions, creams or gels are useful for the topical application of a combination of the invention. The carrier itself may be inert or may possess dermatological benefits of its own. Preferably, the carrier is physically and chemically compatible with a β-amyloid peptides, oligomers, and analogs thereof composition of the invention described herein, and does not unduly impair stability, efficacy or other use benefits associated with the compositions of the present invention.

Alternatively, the compounds of the invention are provided as oral compositions (e.g., tablets, capsules, liquids, or sublingual formulations).

### Wound Treatment

Antimicrobials of the invention can be used alone or in conjunction with an absorbent and/or adsorbent material for the treatment of wounds. In one embodiment, a composition comprising β-amyloid peptides, oligomers, and analogs thereof is used to treat a surgical site prior to, during, or following surgery. In other embodiments, a composition of the invention comprising β-amyloid peptides, oligomers, and analogs thereof is used to irrigate a wound or other site having a propensity to develop a pathogen infectionl. Materials comprising antimicrobials of the invention are suitable for absorbing fluids that are contaminated with or are susceptible to contamination by a microbe, such as bodily fluids, secretions, or excretions. Bodily fluids, secretions, or excretions include, but are not limited to, blood, urine, saliva, serous fluid, synovial fluid, gastric secretions, cerebrospinal fluid, sweat, tears, bile, chyme, mucous, vitreous humor, lymph, wound exudate, feces, blood (e.g., menstrual blood), and semen. In one embodiment, an antimicrobial composition of the invention is incorporated into an absorbent fibrous or non-fibrous material suitable for use as a wound dressing, a medical sponge, a hemostatic article, a hemostatic article for the nose, an adhesive bandage, a wound packing, an internal vascular closure packing, an external vascular closure dressing, a swellable absorbent article, or a fibrotic wound packing article.

### Methods for Assaying Antimicrobial Activity

Specific dosage and administration regimens are determined empirically as described herein. Methods for determining the anti-microbial activity of a composition of the invention are known in the art. In one embodiment, antimicrobial activity is estimated by determining the number of microbes that survive incubation with the candidate anti-microbial using an assay for colony forming units. Such methods are described, for example, by Datta et al., Appl Environ Microbiol. 1997 October; 63(10): 4123-4126, Rhee et al., Appl Environ Microbiol. 2003 May; 69(5): 2959-2963; or by determining the effect of an antimicrobial on a bacterial colony of L. monocytogenes, as measured in an inhibitory halo assay (Dieuleveux et al., Appl Environ Microbiol. 1998 February; 64(2): 800-803). In one embodiment, a microscale assay is used (Barreteau et al., “A rapid method for determining the antimicrobial activity of novel natural molecules,” J Food Prot. 2004 September; 67(9):1961-1964).

### Prophylactics and Therapeutics

For therapeutic uses, the β-amyloid peptides, oligomers, and analogs thereof disclosed herein may be administered topically. Other routes of administration include, for example, nasal, local, subcutaneous, intramuscular, or intradermal injections that provide continuous, sustained levels of the drug in the patient. Treatment of human patients or other animals will be carried out using a therapeutically effective amount of a combination therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the skin condition. Generally, amounts will be in the range of those used for other agents used in the treatment of skin conditions associated with acne, although in certain instances lower amounts will be needed because of the increased specificity of the compound. A compound is administered at a dosage that controls the clinical or physiological symptoms of a skin condition as determined by a diagnostic method known to one skilled in the art.

Therapeutic compounds and therapeutic combinations are administered in an effective amount. In certain embodiments, compounds of the invention, such as those described herein, are administered at dosage levels of about 0.1 ppm to about 3000 ppm. In particular embodiments, the β-amyloid peptides, oligomers, and analogs thereof are present at about 0.1, 0.2, 0.5, 0.75, 1, 2, 3, 5, 10, 15, 20, 25, 50, 75, 100, 200, 250, 300, 350, 400, 500, 750, 1000, 1250, 1500, 2000, 2500, and 3000 ppm. Other compounds of the invention are administered in an amount of about 0.0001 to 4.0 grams once per day (or multiple doses per day in divided doses) for adults. Thus, in certain embodiments of this invention, a compound herein is administered at a dosage of any dosage range in which the low end of the range is any amount between 0.1 mg/day and 400 mg/day and the upper end of the range is any amount between 1 mg/day and 4000 mg/day (e.g., 5 mg/day and 100 mg/day, 150 mg/day and 500 mg/day, 300 mg/day-1000 mg/d (oral)). In other embodiments, a compound herein, is administered at a dosage range in which the low end of the range is any amount between 0.1 ppm/day and 4999 ppm/day and the upper end of the range is any amount between 0.2 ppm/day and 5000 ppm/day. Preferably, a combination of the invention is administered topically once per day in a composition comprising at least about 1% β-amyloid peptides, oligomers, and analogs thereof. The dosing interval can be adjusted according to the needs of individual patients. For longer intervals of administration, extended release formulations can be used.

### Formulation of Pharmaceutical Compositions

The administration of a compound for the treatment of a skin disease or disorder may be by any suitable means that results in a concentration of the therapeutic that, combined with other components, is effective in ameliorating, reducing, or stabilizing a skin infection. The compound may be contained in any appropriate amount in any suitable carrier substance, and is generally present in an amount of 0.1-95% by weight of the total weight of the composition. Preferably, the composition is provided in a dosage form that is suitable for topical administration. In other embodiments, the composition is provided in a form that is suitable for oral administration. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations. For some applications, controlled release formulations obviate the need for frequent dosing during the day. Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Topical Administration

Topical administration of the pharmaceutical compositions of this invention is especially useful for preventing or treating a skin disease or disorder or for enhancing the appearance of skin. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water.

### Therapeutic Combinations

If desired, compositions comprising β-amyloid peptides, oligomers, and analogs thereof are administered alone or in combination with other therapeutics commonly used for the treatment of skin infections. In one embodiment, a composition of the invention comprises one or more of doxycycline, tetracycline, erythromicin, minocycline, trimethoprim, azithromycin, and/or clindamycin. If desired a composition of the invention is administered together with an inert carrier suitable for topical administration, including but not limited to, cocamidopropyl betaine, methylparaben, butylene glycol, benzoic acid, octoxynol-9, and/or urea.

### Subject Monitoring

The disease state or treatment of a subject having a skin disease or disorder can be monitored during treatment with a composition of the invention. Such monitoring may be useful, for example, in assessing the efficacy of a particular agent in a patient. therapeutics that promote skin health or that enhance the appearance of skin are taken as particularly useful in the invention.

### Cleaning Compositions

Anti-microbial compositions containing β-amyloid peptides, oligomers, and analogs thereof may be employed in the form of aqueous or non-aqueous dilutions for application and used to sanitize a surface. By “sanitize” is meant prevent or treat microbial contamination of the surface. Desirably, compositions of the invention reduce microbial contaminants in the inanimate environment to levels considered safe according to public health ordinance. An anti-microbial of the invention may formulated as an aerosol, mist, spray, foam, liquid, wash, rinse, or as a bath to treat submerged items, articles or surfaces, and/or added to an aqueous system to treat submerged surfaces. Thus, a wide variety of suitable application methods include for example, but are not limited to, pouring, spraying, application with a trigger sprayer, aerosol sprayer or device containing a pressurized propellant and/or condensed gas, spraying onto a surface from a container attached to a hose, wiping onto a surface with a pre-moistened disposable device such as for example, but not limited to, a nonwoven wipe, cloth and/or sponge wetted with the inventive compositions. Suitable application methods include any method in which the inventive compositions are applied directly in either neat form, or concurrent with and/or following dilution of a concentrated composition with a suitable aqueous diluent, such as for example water.

In one representative embodiment, β-amyloid peptides, oligomers, and analogs thereof are formulated as concentrated mixtures of ingredients, optionally including a first aqueous or non-aqueous diluent (e.g., ethanol) that may be further diluted to prepare a ready-to-use solution. During use, and in order to sanitize or disinfect a surface, the inventive compositions are applied to and allowed to contact the surface for a proscribed time to effect microbial reduction or kill. After this contact time, the inventive compositions may be allowed to remain in place, or may optionally be wiped or removed from the surface by some suitable means. Optionally, the treated surface can be rinsed with water to remove the inventive compositions following treatment. Compositions of the invention may optionally include cleaning agents and other adjuncts and hence provide simultaneous cleaning and antimicrobial treatment of surfaces.

The antimicrobial compositions can be used in a household, commercial, restaurant, medical, business and/or outdoor environment. Surfaces to which the antimicrobial composition may be applied include, but are not limited to those made from metal, plastic, stone, glass, ceramic, painted surfaces, wallpaper, textiles, carpets, and the like. Antimicrobials of the invention may be used to sanitize any of the following: sink, tile, bathtub, shower wall, toilet bowl, kitchen countertop, tabletop, table covering, cutting board, eating utensil, stove top, oven, microwave oven, refrigerator, wall, floor, and/or window. Such surfaces are commonly found in households, hospitals, and food production facilities. In addition, the compositions can be used on the interior and exterior surfaces of common objects of construction, including, but not limited to exterior and interior surfaces of an airplane, automobile, bathtub, boat, building, fluid distributing system, household-appliance, household fixture, shower stall, sink, ship, sanitary closet, vehicle, water distribution system, water recirculation system, and/or combinations thereof, and further including the finished, laminated, coated and/or painted surfaces thereof.

If desired, compositions of the invention used as cleaners or sanitizers are provided in combination with an effective amount of one or more surfactants. Such surfactants include, but are not limited to, nonionic, semi-polar, anionic, cationic, zwitterionic, and/or amphoteric surfactants. In one aspect of this embodiment, the surfactant includes, but is not limited to, lauryl sulfate, laurylether sulfate, cocamidopropylbetaine, alkyl polyglycosides, and/or amine oxides. The surfactant content in and/or used in combination with the improved cleaning composition is about 0.1-2 weight percent. In yet a further aspect of this embodiment, the surfactant content in and/or used in combination with the improved cleaning composition is about 0.15-1.5 weight percent In still yet a further aspect of this embodiment, the surfactant content in and/or used in combination with the improved cleaning composition is about 0.2-1.5 weight percent. In another aspect of this embodiment, the surfactant content in and/or used in combination with the improved cleaning composition is about 0.2-1.25 weight percent. In yet another aspect of this embodiment, the surfactant content i is about 0.5-1.25 weight percent. In still another aspect of this embodiment, the surfactant content is about 0.1-1 weight percent. In still yet another aspect of this embodiment, the surfactant content is about 0.15-0.8 weight percent. In a further aspect of this embodiment, the surfactant content is about 0.2-0.4 weight percent. In yet a further aspect of this embodiment, the surfactant content is less than about 0.5 weight percent.

The cleaning or sanitizing composition typically includes water. When the improved cleaning composition is a liquid, water based, ready-to-use cleaner, the water content of the improved cleaning composition is generally over 50 weight percent of the improved cleaning composition. Typically, the liquid ready-to-use improved cleaning composition includes at least about 80 weight percent water; however, higher or lower water contents can be used.

One or more additional anti-microbial compounds can be included in and/or used in combination with the improved cleaning composition to enhance the biocidal efficacy of the cleaning composition. Such anti-microbial compounds include, but are not limited to, glycine, sodium actetate, sorbic acid, sodium dehydroacetate, sodium lactate, sodium benzoate, p-hydroxy benzoate, ε-polylysine, milt protein, lysozyme, or any plant derived antimicrobial components, alcohols, peroxides, boric acid and borates, chlorinated hydrocarbons, organometallics, halogen-releasing compounds, mercury compounds, metallic salts, pine oil, essential oils, organic sulfur compounds, iodine compounds, silver nitrate and other silver compounds, quaternary phosphate compounds, and/or phenolics.

### Kits

The invention provides kits for the treatment or prevention of a skin disease or disorder, or symptoms thereof. In one embodiment, the kit includes a pharmaceutical pack comprising an effective amount of a β-amyloid peptides, oligomers, and analogs thereof. Preferably, the compositions are present in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing a skin disease or disorder. The instructions will generally include information about the use of the compounds for the treatment or prevention of a skin disease or disorder. In other embodiments, the instructions include at least one of the following: description of the compound or combination of compounds; dosage schedule and administration for treatment of a skin condition associated with acne, dermatitis, psoriasis, or any other skin condition characterized by inflammation or a bacterial infection, or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Aβ Peptides Possess Antimicrobial Activity

Antimicrobial activity against a particular microorganism is measured in vitro by a peptide's minimal inhibitory concentration (MIC), which is defined as the lowest concentration able to visibly inhibit growth overnight. The MICs of synthetic LL-37, Aβ40, and Aβ42, against a panel of clinically relevant organisms (Table 1).

TABLE 1
Table 1: The antimicrobial activity of synthetic Aβ1-42
(Aβ42), Aβ1-40 (Aβ40), LL-37 (LL-37), reverse
Aβ42-1 (rAβ42), or scrambled Aβ42 (scAβ42)
peptides were determined as minimal inhibitory concentrations
(MIC) against 12 microorganisms.

| | MIC (μg/ml) | | | | | |
|---|---|---|---|---|---|---|
| Organism | Aβ42 | Aβ40 | roAβ42 | LL-37 | reAβ42 | scAβ42 |
| Candida albicans | 0.78 | 0.78 | 0.78 | 6.25 | >25 | >50 |
| Escherichia coli | 1.56 | 1.56 | 3.13 | 1.56 | >50 | >50 |
| Staphylococcus | 3.13 | 50 | 3.13 | 25 | >50 | >50 |
| epidermidis | | | | | | |
| Streptococcus | 6.25 | 12.5 | 6.25 | 1.56 | 50 | >50 |
| pneumoniae | | | | | | |
| Staphylococcus | 6.25 | 25 | 12.5 | 6.25 | >50 | >50 |
| aureus | | | | | | |
| Listeria | 6.25 | 25 | 6.25 | 25 | >50 | 50 |
| monocytogenes | | | | | | |
| Enterococcus | 6.25 | 50 | 3.13 | 6.25 | 50 | >50 |
| faecalis | | | | | | |
| Streptococcus | 12.5 | 50 | >50 | 12.5 | >50 | >50 |
| agalactiae | | | | | | |
| Pseudomonas | >50 | >50 | >50 | 6.25 | >50 | >50 |
| aeruginosa | | | | | | |
| Streptococcus | >50 | >50 | >50 | 6.25 | >50 | >50 |
| pyogenes | | | | | | |
| Streptococcus | >50 | 50 | >50 | 6.25 | >50 | >50 |
| mitis | | | | | | |
| Streptococcus | >50 | >50 | >50 | 50 | >50 | >50 |
| salivarius | | | | | | |

Antimicrobial activity was assayed by broth microdilution susceptibility test on 96-well plates with microbial growth in wells determined by visual inspection following an overnight incubation. Inhibition of growth in plate wells was confirmed by alamar blue cell viability assay and by surface plating of incubants on agar and counting CFU. Inoculums contained mid-logarithmic phase cells. Consistent with antimicrobial activity specific to the Aβ sequence, inhibition was not observed for reverse and scrambled peptides.

The antimicrobial activity of Aβ peptides was equivalent to or greater than LL-37 for seven of the pathogens tested. These data indicate that Aβ is a bona fide AMP with potencies similar to, or, in some cases surpassing those of LL-37. The synthetic Aβ peptides demonstrated antibiotic activity against Gram-negative and Gram-positive bacteria and the yeast C. albicans. Activity was isoform-specific for six organisms with Aβ42 showing greater potency compared to Aβ40. Equivalent findings were observed for recombinant Aβ42, material that is free of the potentially toxic contaminants associated with conventional solid-phase peptide synthesis. Rodent Aβ42 also demonstrated antimicrobial activity. However, microbial growth was not inhibited by reverse (rAβ42) or scrambled (scAβ42) negative control peptides, thus confirming the antimicrobial action is peptide-specific.

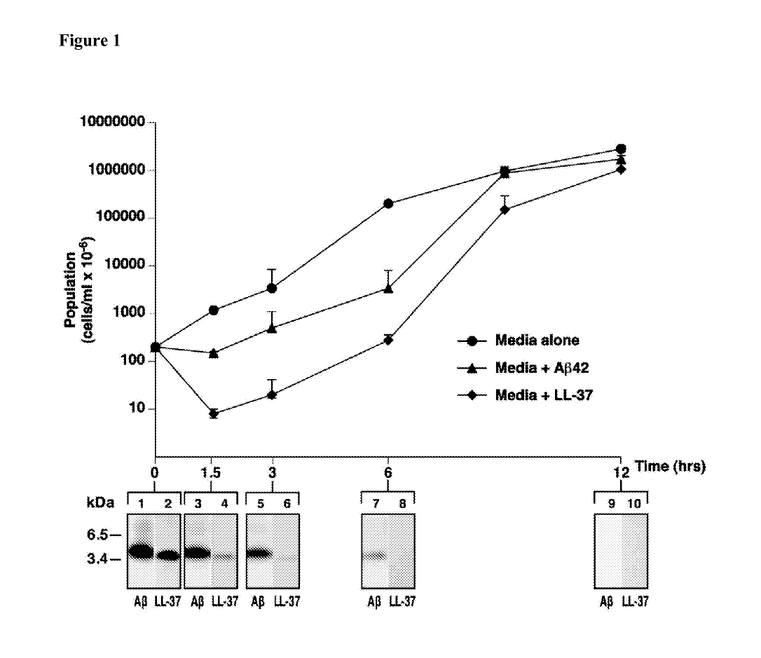

AMPs, including LL-37 [21], can be bacteriostatic or bactericidal depending on peptide concentration, ionic strength, and the type of stressor a colony has previously encountered. The growth curves for E. faecalis in the presence of Aβ42 suggest a predominantly bacteriostatic action for the peptide against this organism under our incubation conditions (FIG. 1). Consistent with previous studies, LL-37 showed potent bactericidal activity against E. faecalis. Microbial growth resumed at later time points, most likely due to degradation of LL-37 and Aβ by protective bacterial proteases (FIG. 1).

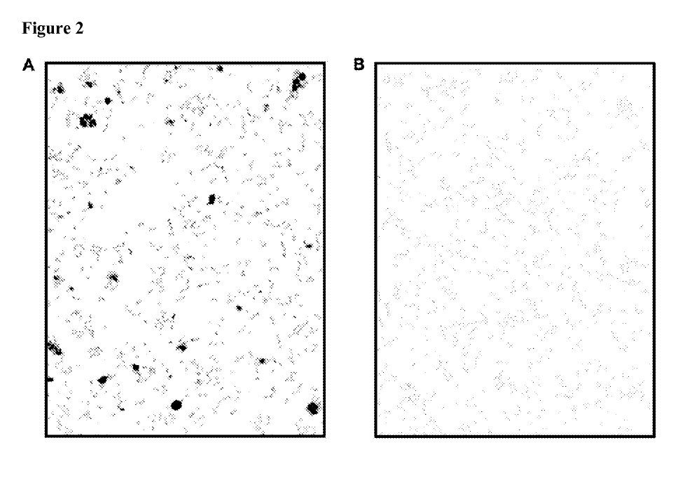

The capacity to associate with microbial lipid bilayers is considered a definitive feature of AMPs [22]. Most antimicrobial peptides are cationic to facilitate binding to anionic bacterial membranes. However, Aβ peptides are anionic under physiological conditions [23]. Nonetheless, data from light microscopic examination of immunostained bacteria pre-incubated with Aβ confirm that the peptide binds to the surface of bacterial cells (FIG. 2). Binding of Aβ to bacterial membranes is consistent with previous studies showing that Aβ readily binds and disrupts negatively charged synthetic lipid bilayers [24,25] and anionic mitochondria membranes [26,27,28], believed to have been originally derived from bacterial membranes.

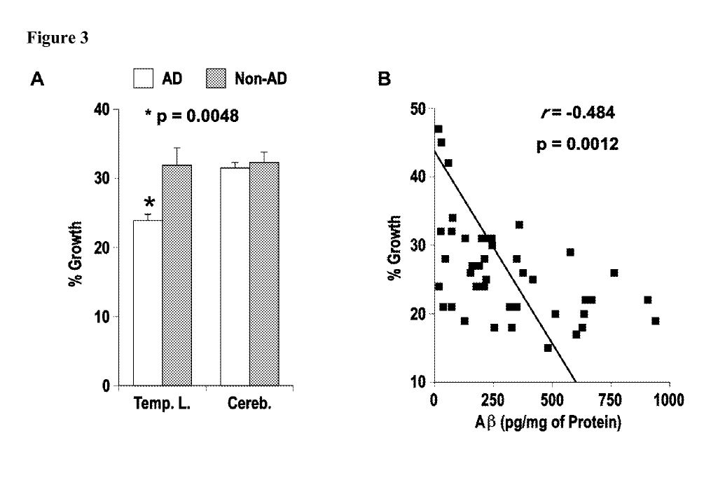

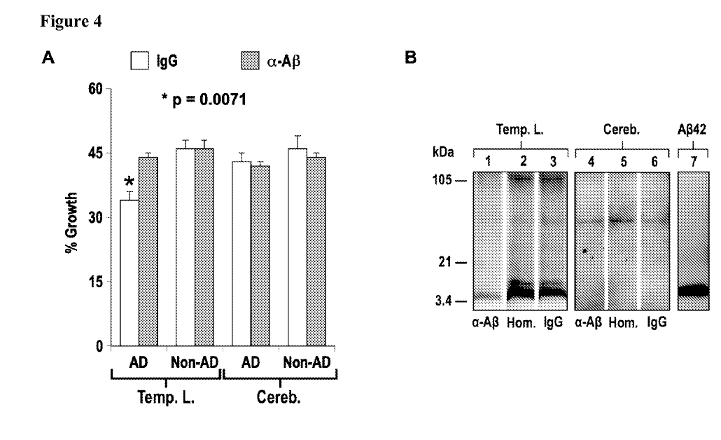

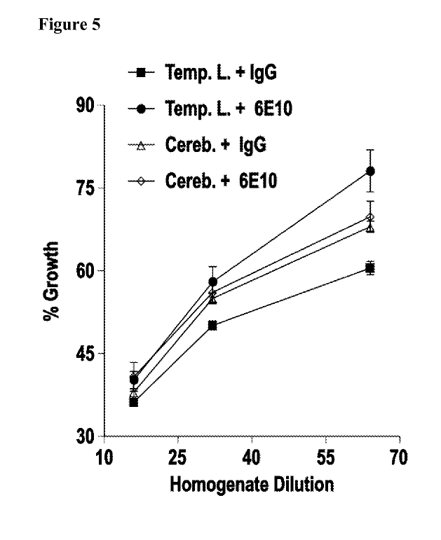

The next experiments tested whether the antimicrobial activity observed for synthetic peptides in vitro could be identified in temporal lobe and cerebellum from human brain. Typically β-amyloid load is high in AD temporal lobe and low in cerebellum. Tissue taken from AD (n=32) or age matched control subjects (n=13) were homogenized and normalized for protein. Aβ40 and Aβ42 levels in brain homogenates were determined by ELISA. Homogenates were then diluted into culture broth and inoculated with Candida albicans. Growth of C. albicans was determined using a fluorescence-based alamar blue microplate assay previously described for following cell viability with this organism [29]. AD temporal lobe homogenates inhibited the growth of C. albicans significantly more (p=0.0048) than non-demented control samples (FIG. 3A). Consistent with an Aβ-mediated antimicrobial activity in AD temporal lobe homogenates, a significant difference in C. albicans growth was not observed with cerebellum samples, which carry a considerably lower Aβ load. Also consistent with Aβ-mediated antimicrobial activity, C. albicans growth significantly correlated with Aβ concentration in temporal lobe homogenates (FIG. 3B), but not in cerebellum samples with Pearson's correlation coefficients (r) of −0.484, p=0.0012 and −0.091, p=0.56, respectively. In addition, the increased antimicrobial activity of AD temporal lobe samples could be significantly attenuated (p=0.0007) by immunodepletion of homogenates with anti-Aβ antibodies (FIG. 4A), consistent with an Aβ-mediated antimicrobial activity in AD brain. Analysis of immunodepleted homogenates confirmed Aβ levels were attenuated in samples incubated with rabbit anti-Aβ antibody (FIG. 4B). Additional experiments confirmed that antimicrobial activity in AD temporal lobe homogenates is also attenuated following immunodepletion with the anti-Aβ mouse monoclonal antibody 6E10 (FIG. 5).

Aβ peptides inhibited the growth of eight of 12 clinically important pathogens screened (Table 2), including the bacteria S. pneumoniae, which is a leading cause of bacterial meningitis [30], and C. albicans, the most common cause of neurocandidiasis [31]. If the normal function of Aβ is to function as an AMP, then an absence of the peptide may result in increased vulnerability to infection. Such an association has been shown for LL-37 and the disorder morbus Kostmann in which patients deficient in this AMP cannot mount an effective defense against pathogens [32]. A relationship between human immunodeficiency and low Aβ levels has not been investigated. However, knockout mice that lack the proteases that generate endogenous rodent Aβ appear to have increased susceptibility to pathogens [33]. BACE1 knockout (KO) mice that generate low levels of Aβ and BACE1- and BACE2-deficient double KO mice, which do not express Aβ, have mortality rates of 40 and 60 percent, respectively. Housing the animals in a pathogen-free environment restores survival rates to that of wild-type mice (>95 percent). The etiology of the immunodeficiency has been investigated but not identified. Adaptive immune responses to vesicular stomatitis virus are the same for BACE-KO and wild-type mice. In addition, markers for adaptive immune system function are normal in BACE-KO mice, including leukocytes migration into the peritoeum following thioglycolateacute-induced acute peritonitis and T-cell cytotoxiocity towards non-host cells. More recently, in a clinical trial of the Aβ42-lowering agent tarenflurbil patients receiving the drug have significantly increased rates of infection [34]. Increased pathogen susceptibility of apparently adaptive immunocompetent BACE-KO mice and AD patients with suppressed Aβ expression is consistent with our finding that Aβ may have a normal protective function as an antimicrobial peptide of the innate immune system.

The immunostatus of APP knockout (APP-KO) mice has yet to be characterized. APP is a member of a larger protein family that includes the amyloid protein precursor-like proteins 1 and 2 (APLP1 and APLP2) [35,36]. APP and APLP proteins appear to have overlapping and partially redundant functions [37,38,39] and share processing pathways, including BACE-mediated generation of APLP-derived peptides analogous to Aβ [37,38,39,40]. It is unclear to what degree APLP-derived proteins may compensate for deficiencies associated with low APP expression. Mice lacking both APP and APLP proteins (triple APP/APLP KO-mice) show early postnatal mortality with severe developmental abnormalities [41,42,43,44,45].

Interestingly, local cortical dysplasias can be infection-mediated and are observed in 68% of triple APP/APLP KO-mice [45]. Partial penetrance is also suggestive of an environmental component in this ectopia.

Recent studies have shown that while the adaptive immune system has limited access to the brain, the CNS can still mount a robust response to invading pathogens via antimicrobial peptides and the innate immune system. Numerous innate immune molecules with potent antimicrobial activity are found in brain, including the recently identified chromogranins [46], neuropeptides neurokinin-1, enkelytin and peptide B, neuropeptide Y, polypeptide tyrosine-tyrosine, and the peptide hormones α-melanocyte stimulating hormone, adenoregulin, adrenomedullin and proadrenomudullin, corticostatin RK-1, neurotensin, and bradykinin [47]. Consistent with an antimicrobial role for brain generated Aβ, AD temporal lobe homogenates contained an average of 24% greater activity against C. albicans than samples from non-AD subjects (FIG. 3A). Furthermore, higher Aβ levels in temporal lobe samples correlated with increased inhibition of C. albicans (FIG. 3B) while immunodepletion of Aβ from AD brain homogenates restored antimicrobial activity to levels equivalent to those of control homogenates (FIGS. 4A and 5). Immunoblot analysis confirmed attenuated Aβ levels in anti-Aβ antibody immunodepleted samples and low β-amyloid load in cerebellum tissue (FIG. 4B). These data support a protective role for Aβ under the conditions found in the brain milieu even though in vivo concentrations of soluble peptide are substantially lower than levels in experiments using synthetic peptide [48]. Several factors may contribute to this apparent discrepancy. First, synergistic AMP interactions in vivo potentiate antimicrobial activity [49]. This effect has been demonstrated for CRAMP (rodent LL-37), for which peptide levels in rodent CNS do not approach concentrations that are needed to obtain positive signals in in vitro assays. However, rat brain extracts depleted of CRAMP have substantially attenuated antimicrobial activity [50]. Moreover, mutant mice lacking CRAMP are more susceptible to CNS infection by meningococcal meningitis [11]. Second, AD brain contains a large pool of neurotoxic oligomeric Aβ species [51,52]. Oligomerization plays a key role in the targeting and permeabilization of bacterial membranes by AMPs [19,53,54]. Neurotoxic oligomeric Aβ species present in AD brain may enhance the antimicrobial activity of homogenates beyond that predicted from in vitro experiments, which add synthetic monomeric peptides to microbial cultures.

A large body of data supports a central role for neuroinflammation in AD neuropathology [55]. A number of studies have proposed Aβ as the source of AD-associated inflammation [56]. However, a re-evaluation of the role of Aβ in inflammation may now be warranted in view of these data suggesting that the peptide functions as an AMP in tissues. Inflammatory response in the immunologically privileged CNS is mediated by the innate immune system. Rather than Aβ acting as a sole independent initiator of neuroinflammation, our data raise the possibility that the peptide may be part of a response mounted by the innate immune system. Thus, Aβ may be one of a family of AMPs known to contribute pro-inflammatory activities under disease conditions. At least one other disease has been shown to involve deposition of an AMP as amyloid, corneal amyloidosis. In corneal amyloidosis the widespread and well-characterized antimicrobial protein lactoferrin accumulates in the subepithelium as insoluble amyloid [57,58]. Semenogelin-derived antimicrobial peptides [59] are also deposited as seminal vesicle amyloid [60] in a common sub-clinical pathology found in elderly men [61].

A number of studies have reported that the CNS of AD patients is infected with pathogens including Chlamydia pneumoniae [62], Borrelia spirochetes [63], Helicobacter pylori [64], and HSV [65]. Deposition of β-amyloid has also been reported for acquired immunodeficiency syndrome patients with brain HIV infection [66]. Given the known genetic influence on Aβ accumulation, genetic factors may contribute to activation of the innate immune system by regulating Aβ production and clearance. At one of the end of the spectrum of known AD genes, highly penetrant mutations such as those in the early-onset familial AD genes, APP, PSENI, and PSEN2, would constitutively trigger cerebral Aβ accumulation with no need for activation of the innate immune system [67]. At the other end of the spectrum, consistent with the increase risk of AD associated with the ε4 variant of the apolipoprotein E gene [68], carriers of the ε4 allele are reported to have higher rates of CNS infection for several of these pathogens [69]. Finally, in a recent family-based genome-wide association scan for late-onset AD, one of four genes achieving genome-wide significance for association with AD was a homologue of CD33, a lectin involved in the innate immune system [70].

While dozens of diseases have been suggested to involve immune abnormalities, for most, the underlying cause of the aberrant immunoresponse remains unclear. For AD, traumatic brain injury [71], stroke [72] and certain forms of inhalant anesthetics [73] have been linked to increased cerebral Aβ levels. While an infection-mediated pathological mechanism for AD is one possibility, this remains to be investigated, and other non-microbial factors may also be involved. Interestingly, peptides containing the microtubule binding sites on tau proteins have also been shown to harbor antimicrobial properties [74].

The capacity to associate with lipid bilayers is considered a definitive feature of AMPs, and the peptides usually affect their antimicrobial activity by membrane permeabilization [75]. Membrane disruption is also thought to be a mechanism for Aβ-mediated cytotoxicity [24,26]. The finding that bacterial membranes stain positive for Aβ following incubation with the peptide (FIG. 2) is consistent with a mechanism that involves association with microbial lipid bilayers. While most AMPs are cationic, Aβ peptides are anionic. Repulsive electrostatic forces between anionic peptides and electronegative phospholipids in bacterial membranes potentially limit antimicrobial activity of this class of AMP. However, in addition to our data, previous studies have conclusively shown that Aβ readily binds and disrupts both synthetic anionic lipid bilayers [24] as well as mitochondrial membranes [26]. Interestingly, mitochondria are thought to be of endosymbiont origin and have anionic membranes that resemble the lipid bilayers of bacteria. A number of AMPs, including LL-37, appear to target and disrupt the mitochondrial membranes of parasitic protozoans [8]. Recent studies have also identified a number of anionic mammalian peptides with antimicrobial activity, including CNS neuropeptides [76] and peptide hormones [47]. Structural studies on the important epithelial anionic AMP dermicidin have shown that an overall positive charge is not a prerequisite for binding of bacterial membranes [77]. Rather, the key modulators of lipid bilayers/peptide association are the peptides charge distribution and secondary conformation. Collectively, these data indicate that AMP activity is not limited to cationic species and that anionic peptides such as Aβ can readily bind bacterial membranes and act as potent antimicrobial agents.

In E. faecalis cultures, Aβ was more resistant to bacterial-mediated degradation than LL-37 (FIG. 1). Bacterial defense mechanisms secrete proteases that target positively charged peptides. Anionic AMPs are believed to be, at least in part, a host counter measure to bacterial resistance mechanisms [78]. Oligomerization is also thought to protect AMPs from microbially-mediated degradation, and Aβ oligomers have been shown to be highly protease resistant. An anionic charge and propensity to oligomerize may therefore help render Aβ resistant to bacterial attack.

AMPs cytotoxicity is usually highly specific for microbes. However, AMPs can also be cytotoxic to select host cells under physiological conditions. Host cell cytotoxicity has been shown for LL-37 [14] which, like Aβ [79], is cytotoxic towards vascular smooth muscle cells. AMP host cell cytoxicity often involves disruption of mitochondrial function, an activity reported for both LL-37 [80,81] and Aβ [27]. Thus, neurotoxicity that has been shown for Aβ is consistent with AMP behavior. The role of AMP host cell cytoxicity in disease and defense is unclear. LL-37 cytotoxicity has been implicated in disease pathology [14], but may also have a normal function in antibody-dependent cell cytotoxicity, a host mechanism for the clearance of virus-infected and transformed cells [81]. At present Aβ's host cell cytotoxicity is only associated with disease. Identification of Aβ as an AMP raises the possibility that host cell cytotoxicity, or at least a component of this activity, may also have a role in innate immunity.

In summary, the finding that Aβ is an antimicrobial peptide is the first evidence that the species responsible for amyloidosis may have a normal function. This stands in stark contrast to current models, which assume β-amyloid deposition to be an accidental process resulting from the abnormal behavior of an incidental product of catabolism. These data suggest that increased Aβ generation, and resulting AD pathology, may be a mediated by a response of the innate immune system to a perceived infection. This model has important implications for current and future AD treatment strategies. First, it raises the possibility of preventing amyloidosis from initiating by pre-emptive targeting of pathogens/insults that stimulate the brain's innate immune system. Second, our model identifies the inflammatory pathways of the innate immune system as targets for modulating Aβ generation/accumulation. The target pathways implicated here are downstream of the inflammatory trigger. Thus, this approach would likely be useful independently of the involvement of infectious agents in AD pathology.

The results reported herein were obtained using the following methods and materials.

### Synthetic Peptides

Experiments used Aβ1-40 (Aβ40), Aβ1-42 (Aβ42), scrambled Aβ (scAβ42), Aβ42-1 (rAβ42), LL-37, and scrambled LL-37 (scLL-37) peptides. Aβ and LL-37 peptides were prepared and purified by Dr. James I. Elliott at Yale University (New Haven, Conn.) using solid-phase peptide synthesis. Scrambled LL-37 peptide was from AnaSpec (San Jose, Calif.). Recombinant human Aβ42 (recAβ42) and rodent Aβ42 (roAβ42) were purchased from rPeptide (Bogart, Ga.) and Calbiochem (Gibbstown, N.J.) respectively. Findings for recombinant and SPPS prepared peptides were equivalent in all experiments.

### Brain Samples

Human brains were obtained 12-24 hrs postmortem. At the time of autopsy, one cerebral hemisphere was sectioned and frozen at −70° C. and the other hemisphere was fixed in formalin for histological examination. The clinical diagnosis of AD was confirmed by subsequent histological evidence of amyloid plaques and neurofibrillary tangles. Samples were provided by the Neurobiology Tissue Bank at the Mass General Institute for Neurodegenerative Disease and Massachusetts General Hospital and included temporal lobe and cerebellum from 32 AD patients and 13 non-demented age-matched control subjects.

### Cell Cultures

Bacteria were from the American Type Culture Collection (ATCC, Manassas, Va.) and included Candida albicans ATCC 10231, Escherichia coli ATCC 25922, Staphylococcus epidermidis ATCC 12228, Streptococcus pneumoniae ATCC 49619, Staphylococcus aureus ATCC 25923, Listeria monocytogenes ATCC 19112, Enterococcus faecalis ATCC 29212, Streptococcus agalactiae ATCC 12386, Pseudomonas aeruginosa ATCC 27853, Streptococcus pyogenes ATCC 19615, Streptococcus mitis ATCC 6249, and Streptococcus salivarius ATCC 13419. Bacteria were cultured aerobically in Mueller-Hinton broth (MHB), Brain and Heart Infusion broth (BHIB), or BHIB supplemented with 1% lysed horse blood and plated on Tryptone Soy Agar (TSA) plates containing 5% defibrinated sheep blood. C. albicans was grown in RPMI-1640 medium (Hyclone, Logan, Utah) with 2% glucose buffered (pH 7.0) and 0.165 M MOPS and surface plated on sabouraud dextrose agar plates. Culture conditions for each organism are included in Table 2. Organisms were subcultured for 2 hrs to generate mid-logarithmic growth cultures for use as inoculates in experiments. Media reagents were obtained from Becton, Dickinson and Company (Sparks, Md.).

TABLE 2

| | Gram | ATCC | Growth | Incub. |
|---|---|---|---|---|
| Organism | Stain | No. | Media | (hrs) |
| Staphylococcus aureus | + | 25923 | MHB | 12 |
| Escherichia coli | − | 25922 | BHIB | 12 |
| Listeria monocytogenes | + | 19112 | BHIB | 18 |
| Pseudomonas aeruginosa | − | 27853 | BHIB | 18 |
| Enterococcus faecalis | + | 29212 | BHIB | 18 |
| Staphylococcus epidermis | + | 12228 | BHIB | 18 |
| Streptococcus agalactiae | + | 12386 | BHIB/LHB | 12 |
| Streptococcus pneumoniae | + | 49619 | BHIB/LHB | 12 |
| Streptococcus mitis | + | 6249 | BHIB/LHB | 12 |
| Streptococcus pyogenes | + | 19615 | BHIB/LHB | 18 |
| Streptococcus salivarius | + | 13419 | BHIB/LHB | 12 |
| Candida albicans | | 10231 | RPMI-1640 | 18 |

### Preparation of Inoculum Containing Mid-Logarithmic Phase Cells

Colonies from agar were transferred by sterile loop to growth media and incubated for 2 hrs at 37° C. to achieve a McFarland density of 0.5. Bacteria inoculum cell densities were normalized to 5×10⁵ cells/ml immediately before use photometrically and subsequently confirmed by colony count. Inoculum of C. albicans contained a cell density of 2.5×10³ CFU/ml.

### Peptide Pre-Treatment and Preparation of Stock Solutions

Bulk powdered peptides were first dissolved in 30% trifluoroethanol (TFE) at 1 mg/ml. Five hundred microliter aliquots of the stock solutions were lyophilized and stored under nitrogen at −20° C. Stock solutions at 2 mg/ml were prepared the day of experimentation from the peptide films by solubilizing a second time in either water or 20% TFE. Aβ stocks prepared in water were sonicated and insoluble peptide aggregates pelleted by centrifugation (10 min×16,000 g). Peptide concentrations in stock solutions were determined immediately before use by bicinchoninic acid (BCA) protein assay. The validity of BCA for assaying Aβ peptides has been established previously [82]. For MIC experiments, peptides were serially diluted into growth media. For other experiments stocks were diluted into required working buffers. Experiments included controls for peptide buffer vehicle alone.

### MIC Determination

Peptide antimicrobial activity was determined as minimal inhibitory concentration (MIC) [83]. Experiments identified peptide MIC by broth microdilution susceptibility test in conjunction with CFU and alamar blue assays. Inoculum containing mid-logarithmic phase cells was dispensed into the wells of polypropylene 96-well plates (Fisher, Pittsburgh, Pa.) containing seven two-fold dilutions of test peptide in growth media. Plates were then incubated aerobically overnight (12 to 18 hrs) at 37° C. Peptide MIC was taken as the lowest concentration able reduce cell growth by CFU and alamar blue assays by at least two-fold and which correlated with the visible loss of a growth button on the bottom of microtiter wells. Experiments were repeated a minimum of three times for each organism, and tests included at least three replicates for each assay condition. Experiments included control serial dilutions of buffer vehicle alone.

Note on radial diffusion assays (RDAs). RDAs have been widely used in previous studies to assess AMP antimicrobial activity. However, in our experiments RDAs proved unreliable for testing Aβ antimicrobial activity because the peptide failed to diffuse away from the point of application. Aβ solutions are prone to aggregation, particularly in the presence of even trace amounts of metal, and interaction with contaminates or the media matrix may lead to rapid precipitation of the peptide within the agar. Aβ peptides also appear to irreversibly absorb to the cellulose disks often used as sample reservoirs in RDAs.

CFU assay; Serial dilutions of incubants were prepared and streaked onto the surface of agar. The agar plates were then incubated overnight at 37° C. and colonies forming units counted.

### Alamar Blue Cell Viability Assay

Microbial growth was determined by following the reduction of the synthetic metabolic substrate resazurin (alamar blue) to a fluorescent product by respiratory enzymes in living cells [84]. Alamar blue assay is used in high throughput screens for antimicrobial agents [85] and is available commercially in kit form from Invitrogen. Microbial growth in experiments was assayed with alamar blue kits according to the manufacturer's instructions. Briefly, resazurin reagent was added to microbial cultures (1:10) and samples incubated for 30 or 60 minutes. Fluorescence signal was measured at excitation of 530 nm and emission at 590 nm. Signal was blanked on sterile media. For experiments with brain homogenates, blank wells contained all components as tests but were not inoculated with C. albicans.

### Anti-Aβ Immunostaining of Bacteria

E. faecalis smears were air-dried on Superfrost/Plus microscope slides (Fisher Scientific, Pittsburgh, Pa.) and then heated to kill and fix bacterial cells. Fixed cells were incubated with 3% methanolic hydrogen peroxide for 30 minutes at room temperature to inhibit endogenous peroxidase activity, passed through graded alcohol, and rinsed three times in deionized water and phosphate-buffered saline (PBS). Slides were then incubated with a 1:2,000 dilution of the anti-Aβ monoclonal antibody (mAb) 6E10 (Covance, Princeton, N.J.) in TBST. Following washing, slides were incubated with goat anti-mouse IgG-coupled to HRP (1:200). Detection and localization steps were performed using Vectastain ABC kit and DAB Substrate Kit (Vector Laboratories, Burlingame, Calif.).

### Assaying Antimicrobial Activity in Brain Homogenate

Samples of AD (n=32) or non-demented control (n=13) temporal lobe and cerebellum were homogenized in three volumes of 10 mM phosphate buffer, pH 7.4 by 12 passes in a glass-on-Teflon homogenizer. Homogenates were diluted into RPMI-1640 media to inhibit C. albicans growth by approximately 50 percent (FIG. 5). Samples were then inoculated (2.5×10³ CFU/ml) with mid-logarithmic growth culture of C. albicans and incubated aerobically for 3 hrs at 37° C. in 96-well microplates (100 μl/well). Alamar blue reagent was added to wells (10 μl) and fluorescence measured after 30 and 60 minutes incubation. Signal from test wells was blanked on samples incubanted without C. albicans. Signal from homogenate blanks was equivalent to uninoculated media alone. Samples were assayed in quadruplicate.

### Assaying Aβ in Tissue Homogenates

Aβ40 and Aβ42 in samples were determined using commercially available ELISA kits (Covance, Princeton, N.J.). Brain homogenates were assayed according to the manufacturer's instructions.

### Immunodepletion of Brain Homogenates

MagnaBind goat anti-rabbit IgG beads (Pierce, Ill.) were pre-incubated overnight with the Aβ specific rabbit anti-amyloid β-peptide antibody (Invitrogen, CA) or rabbit IgG then washed repeatedly. Pooled samples were prepared from temporal lobe (30 AD and 12 non-AD) or cerebellum (32 AD and 13 non-AD) homogenates. The pooled brain homogenates were incubated alone or with the antibody loaded beads at 4° C. for 2 hrs. Final incubation conditions were 5 μg of antibody per mg of original tissue (w/w). Beads were pelleted and soluble fraction removed. Fractions were immunoblotted and probed with the Aβ specific mAb 4G8 (Covance, Princeton, N.J.). Analysis confirmed anti-Aβ antibody treated homogenates were depleted of Aβ (FIG. 4B). Soluble fractions were then tested for antimicrobial activity against C. albicans by alamar blue assay.

### Immunoblotting (Western Blotting)

Samples were first resolved by electrophoresis on SDS-PAGE (4-12% Bis-Tris gels) and then transferred to polyvinylidene fluoride membrane. Membranes were blocked with bovine serum albumin (10%) then probed with mAb 4G8 (1:200), mAb 6E10 (1:2,000), or mAb anti-LL-37 (1:200) (Hycult Biotechnology, Uden, The Netherlands). Following washing, membranes were incubated with goat anti-mouse IgG-coupled to HRP. Blots were developed with chemiluminescence reagent (Pierce, Rockford 11l.) and signal captured using a VersDoc digital imaging system (BioRad, Hercules, Calif.). Blot incubations used Tris buffered saline, pH 8 containing 0.1% Tween (TBST).

### Statistical Analysis

Association coefficients between Aβ levels in brain homogenate and C. albicans growth were calculated using Pearson correlation test and linear regression. Experimental groups were compared by unpaired two-tailed t-test with a nominal alpha criterion level of 0.05. Antimicrobial signal in AD and non-AD cohorts passed a D'Agostino-Pearson test for normality (alpha=0.05) with p values of 0.077 and 0.24, respectively. Variances of signal from AD and non-AD cohorts were not significantly different (p=0.18). Alternative non-parametric statistical analysis of antimicrobial activity in temporal lobe homogenates by two-tailed Mann-Whitney U test also returned a significant difference between AD and non-AD cohorts (p=0.018). Statistical analysis used GraphPad Prism software package (La Jolla, Calif.).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Tanzi R E, Moir R D, Wagner SL (2004) Clearance of Alzheimer's Aβ peptide: the many roads to perdition. Neuron 43: 605-608.
2. Le Y, Gong W, Tiffany H L, Tumanov A, Nedospasov S, et al. (2001) Amyloid (β)42 activates a G-protein-coupled chemoattractant receptor, FPR-like-1. J Neurosci 21: RC123.
3. Koldamova R P, Lefterov I M, Lefterova M I, Lazo J S (2001) Apolipoprotein A-I directly interacts with amyloid precursor protein and inhibits Aβ aggregation and toxicity. Biochemistry 40: 3553-3560.
4. Maezawa I, Jin L W, Woltjer R L, Maeda N, Martin G M, et al. (2004) Apolipoprotein E isoforms and apolipoprotein AI protect from amyloid precursor protein carboxy terminal fragment-associated cytotoxicity. J Neurochem 91: 1312-1321.
5. Zlokovic B V, Yamada S, Holtzman D, Ghiso J, Frangione B (2000) Clearance of amyloid β-peptide from brain: transport or metabolism? Nat Med 6: 718-719.
6. Lee M, You H J, Cho S H, Woo C H, Yoo M H, et al. (2002) Implication of the small GTPase Racl in the generation of reactive oxygen species in response to β-amyloid in C6 astroglioma cells. Biochem J 366: 937-943.
7. Paris D, Town T, Parker T A, Tan J, Humphrey J, et al. (1999) Inhibition of Alzheimer's β-amyloid induced vasoactivity and proinflammatory response in microglia by a cGMP-dependent mechanism. Exp Neurol 157: 211-221.
8. Zaiou M (2007) Multifunctional antimicrobial peptides: therapeutic targets in several human diseases. J Mol Med 85: 317-329.
9. Gudmundsson G H, Agerberth B, Odeberg J, Bergman T, Olsson B, et al. (1996) The human gene FALL39 and processing of the cathelin precursor to the antibacterial peptide LL-37 in granulocytes. Eur J Biochem 238: 325-332.
10. Zanetti M (2004) Cathelicidins, multifunctional peptides of the innate immunity. Leukoc Biol 75: 39-48.
11. Bergman P, Johansson L, Wan H, Jones A, Gallo R L, et al. (2006) Induction of the antimicrobial peptide CRAMP in the blood-brain barrier and meninges after meningococcal infection. Infect Immun 74: 6982-6991.
12. Ong P Y, Ohtake T, Brandt C, Strickland I, Boguniewicz M, et al. (2002) Endogenous antimicrobial peptides and skin infections in atopic dermatitis. N Engl J Med 347: 1151-1160.
13. Von Haussen J, Koczulla R, Shaykhiev R, Herr C, Pinkenburg O, et al. (2008) The host defence peptide LL-37/hCAβ-18 is a growth factor for lung cancer cells. Lung Cancer 59: 12-23.
14. Ciornei C D, Tapper H, Bjartell A, Sternby N H, Bodelsson M (2006) Human antimicrobial peptide LL-37 is present in atherosclerotic plaques and induces death of vascular smooth muscle cells: a laboratory study. BMC Cardiovasc Disord 6: 49.
15. Kirkitadze M D, Bitan G, Teplow D B (2002) Paradigm shifts in Alzheimer's disease and other neurodegenerative disorders: the emerging role of oligomeric assemblies. J Neurosci Res 69: 567-577.
16. Wogulis M, Wright S, Cunningham D, Chilcote T, Powell K, et al. (2005) Nucleation-dependent polymerization is an essential component of amyloid-mediated neuronal cell death. J Neurosci 25: 1071-1080.
17. Walsh D M, Klyubin I, Shankar G M, Townsend M, Fadeeva J V, et al. (2005) The role of cell-derived oligomers of Aβ in Alzheimer's disease and avenues for therapeutic intervention. Biochem Soc Trans 33: 1087-1090.
18. Oren Z, Lerman J C, Gudmundsson G H, Agerberth B, Shai Y (1999) Structure and organization of the human antimicrobial peptide LL-37 in phospholipid membranes: relevance to the molecular basis for its non-cell-selective activity. Biochem J 341 (Pt 3): 501-513.
19. Sood R, Domanov Y, Pietiainen M, Kontinen V P, Kinnunen P K (2008) Binding of LL-37 to model biomembranes: insight into target vs host cell recognition. Biochim Biophys Acta 1778: 983-996.
20. Turner J, Cho Y, Dinh N N, Waring A J, Lehrer R I (1998) Activities of LL-37, a cathelin-associated antimicrobial peptide of human neutrophils. Antimicrob Agents Chemother 42: 2206-2214.
21. Sieprawska-Lupa M, Mydel P, Krawczyk K, Wojcik K, Puklo M, et al. (2004) Degradation of human antimicrobial peptide LL-37 by staphylococcus aureus-derived proteinases. Antimicrob Agents Chemother 48: 4673.
22. Bechinger B, Lohner K (2006) Detergent-like actions of linear amphipathic cationic antimicrobial peptides. Biochim Biophys Acta 1758: 1529-1539.
23. Tomski S J, Murphy R M (1992) Kinetics of aggregation of synthetic β-amyloid peptide. Arch Biochem Biophys 294: 630-638.
24. Chauhan A, Ray I, Chauhan V P (2000) Interaction of amyloid β-protein with anionic phospholipids: possible involvement of Lys28 and C-terminus aliphatic amino acids. Neurochem Res 25: 423-429.
25. Chi E, Ege C, Winans A, Majewski J, Wu G, et al. (2008) Lipid membrane templates the ordering and induces the fibrillogenesis of Alzheimer's disease amyloid-β peptide. Proteins 72: 1-24.
26. Rodrigues C M, Sold S, Silva R, Brites D (2000) Bilirubin and amyloid-β peptide induce cytochrome c release through mitochondrial membrane permeabilization. Mol Med 6: 936-946.
27. Eckert G P, Wood W G, Müller W E (2001) Effects of aging and β-amyloid on the properties of brain synaptic and mitochondrial membranes. Journal of Neural Transmission 108: 1051-1064.
28. Chen X, Stern D, Yan S D (2006) Mitochondrial dysfunction and Alzheimer's disease. Curr Alzheimer Res 3: 515-520.
29. Repp K K, Menor S A, Pettit R K (2007) Microplate Alamar blue assay for susceptibility testing of Candida albicans biofilms. Med Mycol 45: 603-607.
30. Leib S L, Tauber M G (1999) Pathogenesis of bacterial meningitis. Infect Dis Clin North Am 13: 527-548, v-vi.
31. Chakrabarti A (2007) Epidemiology of central nervous system mycoses. Neurology India 55: 191-197.
32. Pütsep K, Carlsson G, Boman H G, Andersson M (2002) Deficiency of antibacterial peptides in patients with morbus Kostmann: an observation study. Lancet 360: 1144-1149.
33. Dominguez D, Tournoy J, Hartmann D, Huth T, Cryns K, et al. (2005) Phenotypic and biochemical analyses of BACE1- and BACE2-deficient mice. J Biol Chem 280: 30797-30806.
34. Green R C, Schneider L S, Amato D A, Beelen A P, Wilcock G, et al. (2009) Effect of tarenflurbil on cognitive decline and activities of daily living in patients with mild Alzheimer disease: a randomized controlled trial. JAMA 302: 2557-2564.
35. Wasco W, Bupp K, Magendantz M, Gusella J F, Tanzi R E, et al. (1992) Identification of a mouse brain cDNA that encodes a protein related to the Alzheimer disease-associated amyloid 13 protein precursor. Proc Natl Acad Sci USA 89: 10758-10762.
36. Wasco W, Brook J D, Tanzi R E (1993) The amyloid precursor-like protein (APLP) gene maps to the long arm of human chromosome 19. Genomics 15: 237-239.
37. Li Q, Südhof T C (2004) Cleavage of amyloid-β precursor protein and amyloid-β precursor-like protein by BACE 1. J Biol Chem 279: 10542-10550.
38. Pastorino L, Ikin A F, Lamprianou S, Vacaresse N, Revelli J P, et al. (2004) BACE (β-secretase) modulates the processing of APLP2 in vivo. Mol Cell Neurosci 25: 642-649.
39. Jacobsen K T, Iverfeldt K (2009) Amyloid precursor protein and its homologues: a family of proteolysis-dependent receptors. Cell Mol Life Sci 66: 2299-2318.
40. Minogue A M, Stubbs A K, Frigerio C S, Boland B, Fadeeva J V, et al. (2009) gamma-secretase processing of APLP1 leads to the production of a p3-like peptide that does not aggregate and is not toxic to neurons. Brain Res 1262: 89-99.
41. Zheng H, Jiang M, Trumbauer M E, Sirinathsinghji D J, Hopkins R, et al. (1995) β-Amyloid precursor protein-deficient mice show reactive gliosis and decreased locomotor activity. Cell 81: 525-531.
42. Chen Y, Tang B L (2006) The amyloid precursor protein and postnatal neurogenesis/neuroregeneration. Biochem Biophys Res Commun 341: 1-5.
43. Heber S, Herms J, Gajic V, Hainfellner J, Aguzzi A, et al. (2000) Mice with combined gene knock-outs reveal essential and partially redundant functions of amyloid precursor protein family members. J Neurosci 20: 7951-7963.
44. Magara F, Müller U, Li Z W, Lipp H P, Weissmann C, et al. (1999) Genetic background changes the pattern of forebrain commissure defects in transgenic mice underexpressing the β-amyloid-precursor protein. Proc Natl Acad Sci USA 96: 4656-4661.
45. Herms J, Anliker B, Heber S, Ring S, Fuhrmann M, et al. (2004) Cortical dysplasia resembling human type 2 lissencephaly in mice lacking all three APP family members. EMBO J. 23: 4106-4115.
46. Shooshtarizadeh P, Zhang D, Chich J-F, Gasnier C, Schneider F, et al. (2009) The antimicrobial peptides derived from chromogranin/secretogranin family, new actors of innate immunity. Regulatory peptides: In Press.
47. Brogden K A, Guthmiller J M, Salzet M, Zasloff M (2005) The nervous system and innate immunity: the neuropeptide connection. Nat Immunol 6: 558-564.
48. Chemy R A, Legg J T, McLean C A, Fairlie D P, Huang X, et al. (1999) Aqueous dissolution of Alzheimer's disease Aβ amyloid deposits by biometal depletion. J Biol Chem 274: 23223-23228.
49. McCafferty D G, Cudic P, Yu M K, Behenna D C, Kruger R (1999) Synergy and duality in peptide antibiotic mechanisms. Curr Opin Chem Biol 3: 672-680.
50. Bergman P, Termen S, Johansson L, Nystrom L, Arenas E, et al. (2005) The antimicrobial peptide rCRAMP is present in the central nervous system of the rat. J Neurochem 93: 1132-1140.
51. Kuo Y M, Emmerling M R, Vigo-Pelfrey C, Kasunic T C, Kirkpatrick J B, et al. (1996) Water-soluble Aβ (N-40, N-42) oligomers in normal and Alzheimer disease brains. J Biol Chem 271: 4077-4081.
52. Roher A E, Chaney M O, Kuo Y M, Webster S D, Stine W B, et al. (1996) Morphology and toxicity of Aβ-(1-42) dimer derived from neuritic and vascular amyloid deposits of Alzheimer's disease. J Biol Chem 271: 20631-20635.
53. Sal-Man N, Oren Z, Shai Y (2002) Preassembly of membrane-active peptides is an important factor in their selectivity toward target cells. Biochemistry 41: 11921-11930.
54. Glukhov E, Stark M, Burrows L L, Deber C M (2005) Basis for selectivity of cationic antimicrobial peptides for bacterial versus mammalian membranes. J Biol Chem 280: 33960-33967.
55. Tuppo E E, Arias H R (2005) The role of inflammation in Alzheimer's disease. Int Biochem Cell Biol 37: 289-305.
56. Townsend K P, Obregon D, Quadros A, Patel N, Volmar Ch, et al. (2002) Proinflammatory and vasoactive effects of Aβ in the cerebrovasculature Ann NY Acad Sci 977: 65-76.
57. Ando Y, Nakamura M, Kai H, Katsuragi S, Terazaki H, et al. (2002) A novel localized amyloidosis associated with lactoferrin in the cornea. Lab Invest 82: 757-766.
58. Araki-Sasaki K, Ando Y, Nakamura M, Kitagawa K, Ikemizu S, et al. (2005) Lactoferrin Glu561Asp facilitates secondary amyloidosis in the cornea. Br J Ophthalmol 89: 684.
59. Edstrom A M L, Malm J, Frohm B, Martellini J A, Giwercman A, et al. (2008) The major bactericidal activity of human seminal plasma is zinc-dependent and derived from fragmentation of the semenogelins. J Immunol 181: 3413-3421.
60. Linke R P, Joswig R, Murphy C L, Wang S, Zhou H, et al. (2005) Senile seminal vesicle amyloid is derived from semenogelin I. J Lab Clin Med 145: 187-193.
61. Kee K H, Lee M J, Shen S S, Suh J H, Lee O-J, et al. (2008) Amyloidosis of seminal vesicles and ejaculatory ducts: a histologic analysis of 21 cases among 447 prostatectomy specimens. Ann Diagn Pathol 12: 235-238.
62. Itzhaki R F, Wozniak M A, Appelt D M, Balin B J (2004) Infiltration of the brain by pathogens causes Alzheimer's disease. Neurobiol Aging 25: 619-627.
63. Miklossy J, K is A, Radenovic A, Miller L, Forro L, et al. (2006) β-amyloid deposition and Alzheimer's type changes induced by Borrelia spirochetes. Neurobiol Aging 27: 228-236.
64. Kountouras J, Tsolaki M, Gavalas E, Boziki M, Zavos C, et al. (2006) Relationship between Helicobacter pylori infection and Alzheimer disease. Neurology 66: 938-940.
65. Itzhaki R F, Lin W R, Shang D, Wilcock G K, Faragher B, et al. (1997) Herpes simplex virus type 1 in brain and risk of Alzheimer's disease. Lancet 349: 241-244.
66. Youngsteadt E (2008) Virology. Alzheimer's risk factor also aids HIV. Science 320: 1577.
67. Tanzi R E, Kovacs D M, Kim T W, Moir R D, Guenette S Y, et al. (1996) The gene defects responsible for familial Alzheimer's disease. Neurobiol Dis 3: 159-168.
68. Strittmatter W J, Saunders A M, Schmechel D, Pericak-Vance M, Enghild J, et al. (1993) Apolipoprotein E: high-avidity binding to β-amyloid and increased frequency of type 4 allele in late-onset familial Alzheimer disease. Proc Natl Acad Sci USA 90: 1977-1981.
69. Urosevic N, Martins R N (2008) Infection and Alzheimer's disease: the APOE epsilon4 connection and lipid metabolism. J Alzheimers Dis 13: 421-435.
70. Bertram L, Lange C, Mullin K, Parkinson M, Hsiao M, et al. (2008) Genome-wide association analysis reveals putative Alzheimer's disease susceptibility loci in addition to APOE. Am J Hum Genet. 83: 623-632.
71. Roberts G W, Gentleman S M, Lynch A, Graham D I (1991) 13 A4 amyloid protein deposition in brain after head trauma. Lancet 338: 1422-1423.
72. Tesco G, Koh Y H, Kang E L, Cameron A N, Das S, et al. (2007) Depletion of GGA3 stabilizes BACE and enhances β-secretase activity. Neuron 54: 721-737.
73. Xie Z, Dong Y, Maeda U, Moir R, Inouye S K, et al. (2006) Isoflurane-induced apoptosis: a potential pathogenic link between delirium and dementia. J Gerontol A Biol Sci Med Sci 61: 1300-1306.
74. Kobayashi N, Masuda J, Kudoh J, Shimizu N, Yoshida T (2008) Binding sites on tau proteins as components for antimicrobial peptides. Biocontrol Sci 13: 49-56.
75. Bolintineanu D, Hazrati E, Davis H T, Lehrer R I, Kaznessis Y N (2009) Antimicrobial mechanism of pore-forming protegrin peptides: 100 pores to kill E. coli. Peptides 31: 1-8.
76. Goumon Y, Lugardon K, Kieffer B, Lefevre J, Van Dorsselaer A, et al. (1998) Characterization of Antibacterial COOH-terminal Proenkephalin-A-derived Peptides (PEAP) in Infectious Fluids. Importance of enkelytin, the antibacterial PEAP209-237 secreted by stimulated chromaffin cells. J Biol Chem 273: 29847.
77. Steffen H, Rieg S, Wiedemann I, Kalbacher H, Deeg M, et al. (2006) Naturally processed dermcidin-derived peptides do not permeabilize bacterial membranes and kill microorganisms irrespective of their charge. Antimicrob Agents Chemother 50: 2608-2620.
78. Dennison S R, Morton L H, Brandenburg K, Harris F, Phoenix D A (2006) Investigations into the ability of an oblique alpha-helical template to provide the basis for design of an antimicrobial anionic amphiphilic peptide. FEBS J 273: 3792-3803.
79. Subasinghe S, Unabia S, Barrow C J, Mok S S, Aguilar M I, et al. (2003) Cholesterol is necessary both for the toxic effect of Aβ peptides on vascular smooth muscle cells and for Aβ binding to vascular smooth muscle cell membranes. J Neurochem 84: 471-479.
80. Risso A, Braidot E, Sordano M C, Vianello A, Macrí F, et al. (2002) BMAP-28, an antibiotic peptide of innate immunity, induces cell death through opening of the mitochondrial permeability transition pore. Mol Cell Biol 22: 1926-1935.
81. Aarbiou J, Tjabringa G S, Verhoosel R M, Ninaber D K, White S R, et al. (2006) Mechanisms of cell death induced by the neutrophil antimicrobial peptides alpha-defensins and LL-37. Inflamm Res 55: 119-127.
82. Huang X, Atwood C S, Moir R D, Hartshorn M A, Vonsattel J P, et al. (1997) Zinc-induced Alzheimer's Aβ1-40 aggregation is mediated by conformational factors. J Biol Chem 272: 26464-26470.
83. National Committee for Clinical Laboratory Standards. (2009) Methods for determining bactericidal activity of antimicrobial agents; approved guideline. NCCLS Document M26-A. Wayne, Pa.
84. Shiloh M U, Ruan J, Nathan C (1997) Evaluation of bacterial survival and phagocyte function with a fluorescence-based microplate assay. Infect Immun 65: 3193-3198.
85. Hamid R, Rotshteyn Y, Rabadi L, Parikh R, Bullock P (2004) Comparison of alamar blue and MTT assays for high through-put screening. Toxicol In Vitro 18: 703-710.

1. A method for treating or preventing a microbial infection in a subject in need thereof, the method comprising contacting the subject with an effective amount of a β-amyloid peptide, oligomer, derivative or analog thereof, thereby preventing or inhibiting a microbial infection in the subject.

2. The method of claim 1, wherein the microbial infection comprises a skin infection and the effective amount is an amount sufficient to treat the subject.

3. A method for preventing or inhibiting microbial growth on a surface, the method comprising contacting the surface with an effective amount of a β-amyloid peptide, oligomer, derivative or analog thereof, thereby preventing or inhibiting microbial growth on the surface.

4. The method of claim 3, wherein the method cleans, sanitizes, or disinfects the surface.

5. The method of claim 3, wherein the surface is a wall, floor, ceiling, counter, machine, or other surface or equipment present in an industrial, commercial, or clinical setting.

6. The method of claim 3, wherein the clinical setting is a hospital, medical office, clinic, health center, or laboratory.

7. The method of claim 3, wherein the surface contacted is present in a dwelling, school, office building, plane, train, automobile, restaurant, cafeteria, or daycare center.

8. The method of claim 3, wherein the preventing or inhibiting microbial growth comprises sanitizing or disinfecting and the surface comprises a body part, the method comprising contacting the body part with an effective amount of a β-amyloid peptide, oligomer, derivative or analog thereof, thereby sanitizing or disinfecting the body part.

9. The method of claim 8, wherein the β-amyloid peptide, oligomer, derivative or analog thereof, is formulated as a skin cleanser.

10. The method of claim 8, wherein the skin is cleansed in preparation for surgery.

11. The method of claim 1, wherein the microbe is a bacteria, virus, or fungus.

12. The method of claim 1, wherein the microbe is selected from the group consisting of Candida albicans, Escherichia coli, Staphylococcus epidermidis, Streptococcus pneumoniae, Staphylococcus aureus, Listeria monocytogenes, Enterococcus faecalis, Streptococcus agalactiae Pseudomonas aeruginosa, Streptococcus pyogenes, Streptococcus mitis, and Streptococcus salivariu.

13. The method of claim 1, wherein the effective amount comprises 1% to 90% of a β-amyloid peptide, oligomer, derivative or analog thereof, or combinations thereof.

14. An antimicrobial composition comprising an effective amount of a β-amyloid peptide, oligomer, derivative or analog thereof, thereof in a carrier or diluent.

15. The composition of claim 14, wherein the β-amyloid peptide, oligomer, derivative or analog thereof, is formulated as a water soluble or water insoluble solid, liquid, emulsion, slurry, or powder.

16. The composition of claim 14, wherein the composition is topically administered.

17. A pharmaceutical pack comprising the composition of claim 14 in an individual dosage amount.
